(19) United States
(12) Patent Application Publication
(10) Pub. No.: US 2026/0235567 A1
(43) Pub. Date: Aug. 13, 2026

(54) METHOD FOR DETERMINING A MODIFIED OPERATING STRATEGY FOR A PLURALITY OF SIMILAR GAS SENSORS, AND METHOD FOR OPERATING A GAS SENSOR

(71) Applicant: Robert Bosch GmbH, Stuttgart (DE)

(72) Inventors: Eike Martin Thaden, Hamburg (DE); Philipp Nolte, Gerlingen (DE)

(21) Appl. No.: 19/050,766

(22) Filed: Feb. 11, 2025

(30) Foreign Application Priority Data
Feb. 14, 2024 (DE) 10 2024 201 308.0

(51) Int. Cl. G01N 33/00

(52) U.S. Cl. CPC G01N 33/0075; G01N 33/0006

(57) ABSTRACT

A method for determining a modified operating strategy for a plurality of similar gas sensors. Each of the similar gas sensors is configured to detect measured values for a base number of features according to a regular operating strategy. The method includes: providing measured values detected by the similar gas sensors with a measurement number of features; performing a correlation analysis between the measured values of at least two of the measurement number of the features in each case; checking, based on a result of the correlation analysis, whether a first of the measurement number of the features is correlated with at least a second of the measurement number of the features, at least within specified tolerances; depending on a result of the check, determining a modified operating strategy for the similar gas sensors; and providing the modified operating strategy for use with the similar gas sensors.

### FIELD

The present invention relates to a method for determining a modified operating strategy for a plurality of similar gas sensors, to a method for operating a gas sensor, to a computing unit and a computer program for performing them, and to a gas sensor.

### BACKGROUND INFORMATION

Gas sensors can be used to determine information about gases such as their composition or their concentration, in particular in the case of trace gases and/or odors in air as a carrier gas. Gas sensors that can process various gases (or gas mixtures) are also called multi-gas sensors. They can also be referred to as so-called electronic noses.

### BACKGROUND INFORMATION

According to the present invention, a method for determining a modified operating strategy for a plurality of similar gas sensors, a method for operating a gas sensor, a gas sensor as well as a computing unit and a computer program for performing the methods are provided. Advantageous example embodiments of the present invention are disclosed herein.

The present invention relates to gas sensors, in particular multi-gas sensors. By using a gas sensor, components of a gaseous atmosphere can be determined qualitatively or quantitatively. A gaseous atmosphere comprises a mixture of gases and/or vapors. It can also be predominantly air, which can contain further components in addition to natural components. These individual components can be described, for example, in terms of their chemical substances (elements, compounds) and their concentrations, for example (molar) concentrations of ethanol, carbon monoxide, nicotine or certain refrigerants (e.g., as leakage from cooling systems/vehicle air conditioning systems). This term of gaseous components can also include existing air humidity as a chemical compound. The purpose of the gas sensor can thus in particular be to determine a subset of these chemical substances and their (molar) concentrations. If a typical carrier gas or carrier gas mixture (e.g., air) is used in an application, the relevant information content can be seen in particular in the additional substances; the basic composition of the carrier gas (air) can be assumed to be known per se or is known and no longer relevant in this respect.

In general, this can be referred to as the determination or recognition of a substance or target substance (which itself can comprise a plurality of different components) in a target environment. Target substance and target environment refer to a substance that is to be determined when the gas sensor is later used in the desired environment.

However, the components of the gaseous atmosphere can also be considered in a summarized form, especially in the case of odors. For example, cigarette smoke, perfumes or foods such as beer or coffee can contain a variety of chemical compounds that carry the odor and are not necessarily in a fixed molar ratio that can be reproduced from sample to sample. Here, the purpose of the gas sensor can in particular be to identify the odor type from a set of target odors (e. g., categorically yes/no; or in a semi-quantitative gradation, e.g., on a standardized scale from 0 to 1 depending on the degree of dilution of the emitted odor from the odor carrier with air). Even with this alternative, summarized meaning (without explicitly naming the individual chemical substances), the purpose of the gas sensor can be understood as the determination or recognition of a subset of existing odors as a target substance in the target environment (i.e., for example, the gaseous atmosphere). In this respect, reference may also be made to an odor sensor instead of a gas sensor.

Gas sensors can thus be used to determine information about one or more gaseous components, hereinafter also referred to as target substances, of a gaseous atmosphere or, in general, a target environment depending on the context in particular in terms of chemical substances and molar concentrations or, as is in particular the case with odors in air as a carrier medium, in terms of recognizing the typical odor source. In the case of odors, the information can thus also be evaluated quantitatively, e.g., as a degree of dilution.

For this purpose, the gas sensor can be used to detect measured values in a target environment, i.e., a gaseous atmosphere, from which information about the target substance can then be determined. The gas sensor does not necessarily have to detect all components that become or can be a component of the gaseous atmospheres in an application. The acquisition of information can be limited to the target substance. The other components can also be referred to as disturbing substances or disturbing components, regardless of whether they are specifically identifiable chemical substances or the combination of potentially many chemical substances from one odor carrier.

A machine learning algorithm or a machine learning model can be used to determine the information of target substances or to recognize them from measured values, i.e., for example, to classify and/or quantify odors. A machine learning model can, for example, comprise or be an artificial neural network. The machine learning model then receives the measured values as input and determines the information about the gas or the target substance, which the machine learning model then outputs.

In order to determine the most accurate information possible from the measured values, a machine learning model must be trained. This requires so-called training data, which typically comprise a plurality of measured values of a substance (training substance) in a training environment, i.e., a substance that is later to be recognized as a target substance in a target environment. It is understood that different training substances with corresponding measured values can be used for different target substances to be recognized. Training substance and training environment refer to a substance that is used when collecting training data for the gas sensor in a suitable environment.

So-called supervised learning or training requires not only the measured values (so-called features) themselves but also the corresponding information, so that the machine learning algorithm or its weights and thus the machine learning model can be adapted accordingly, so that the correct information about a target substance is output for certain measured values.

This information is also referred to as annotations or labels and generally comprises at least a description of the training substance. The measured values and the annotations can be linked, namely, via time information or timestamps, i.e., a measured value detected at a specific time point is assigned information as to whether or not the training substance was present in the training environment at that time point.

Such a data set (the training data) is thereby accessible to a machine learning process. A plurality of odors/substances can also be detected in parallel. Depending on the structure of the labels, models can be trained using artificial intelligence methods. As a result, the odors are reproduced in a classified or quantitatively graded manner from sensor features.

Gas sensors or odor sensors can, for example, also be used in networked devices, e.g., for monitoring interior spaces in vehicles or buildings with regard to cleanliness, contamination, cigarette smoke and the like.

Typical gas sensors detect measured values for a certain number of features. Gas sensors based on a thermally modulated sensor are, for example, configured to run through a specified temperature profile during operation and to detect measured values for different features at different temperatures. Alternatively or additionally, for example, measured values can be detected at different time periods since a change in temperature, i.e., before the measured value is detected, waiting takes place for different amounts of time since the temperature was last changed.

For example, a gas sensor can comprise ten different features for which measured values are detected and which are used for the analysis or determination of a substance. In addition, features for pressure, temperature, humidity may be present, for example. During regular operation, i.e., according to a regular operating strategy, measured values for a base number of features are to be detected by means of a gas sensor. In other words, a regular operating strategy thus means that the gas sensor is operated in such a way that it detects measured values for a certain number of features (the base number). For example, the gas sensor can be produced in this way, i.e., as long as the gas sensor is not changed, it is operated with the regular operating strategy.

This base number of features then represents, for example, input values for an already trained machine learning model, which provides the odor information categorically or through concentration values as an inference. In principle, this base number of features can be generated from individual sensors by an electrical mode of operation (e.g., the aforementioned thermal modulation) or from a plurality of individual sensors integrated on the same chip or in completely different electronic modules.

When operating gas sensors or odor sensors in, for example, IoT applications, it may be necessary to centrally evaluate raw data from gas sensors (e.g., from vehicles or buildings). Gas sensors usually involve a variety of signal features, the aforementioned base number of features. However, the regular evaluation of a plurality of sensor features per device is associated with costs and effort, e.g., the energy consumption in the data-collecting sensor unit (e.g., relevant for battery-operated sensor units), the data volume to be transmitted, e.g., via a mobile radio network, and any computing power necessary.

However, instead of a regular operating strategy, a modified operating strategy for gas sensors may be advisable if, for example, it turns out that the application involves a greater or lesser variety of target odors or disturbances than the variety for which the gas sensor was developed, for example on a laboratory basis.

Against this background, within the scope of the present invention, a possibility for determining a modified operating strategy for a plurality of similar gas sensors is proposed, wherein each of the plurality of similar gas sensors in each case is configured to detect measured values for a base number of features according to a regular operating strategy, as mentioned above.

According to an example embodiment of the present invention, for this purpose, measured values detected by the plurality of similar gas sensors with a measurement number of features are provided. In one embodiment, the measurement number can correspond to the base number, as explained in more detail below. A correlation analysis is then performed between the measured values of at least two of the measurement number of the features in each case. In a simple case, this can, for example, comprise a correlation analysis between the measured values of two of the measurement number of the features in each case. However, this simple case can also be just a first step and, in further steps, for example, a multiple correlation analysis can also be performed, namely, between the measured values of, for example, three of the measurement number of features. In further steps, this can then be performed successively with the measured values of, for example, four of the measurement number of features, etc. In general, however, the correlation analysis can, for example, also be performed only between the measured values of three or four of the measurement number of the features in each case.

In one example embodiment of the present invention, the measured values are first examined as to whether one of the base number of the features of the plurality of similar gas sensors is not necessary or whether at least one additional feature might be necessary. This can be carried out as part of a so-called cluster analysis. This may mean in particular that the relationships found suggest that a feature is redundant, i.e., does not provide any additional information, and therefore would not even be necessary during operation; however, it may in particular also be the case that the relationships found indicate, i.e., provide indications, that even more information could be obtained with an additional feature. If all expected odors occur in such a cluster analysis, an in-depth correlation analysis is not necessary. In this case, the correlation analysis is only performed if the examination is positive, i.e., that one of the features is not needed or that an additional feature might be necessary.

According to an example embodiment of the present invention, as part of the correlation analysis, it is then checked whether a first of the measurement number of the features is correlated with at least a second of the measurement number 1 of the features, at least within specified tolerances. At this point, it should be mentioned that, in the case of the aforementioned multiple correlation analysis, a correlation of a first feature with more than one second feature can occur. Depending on a result of the check, a modified operating strategy is then determined for the plurality of similar gas sensors and is provided for use with the plurality of similar gas sensors.

Such a modified operating strategy can, for example, save costs or recognize new odors.

In one example embodiment of the present invention, the measurement number corresponds to the base number, as mentioned above. In this case, if the result of the check is positive, in the modified operating strategy for the plurality of similar gas sensors, an operation for a selected one of the first and the at least one second of the base number of the features is changed. In particular, an originally developed necessary signal feature can thus be identified as unnecessary and omitted again. Nevertheless, a machine learning model trained on the base number of features can continue to be used. This is because, here, the omitted feature can be replaced by the one that correlates with the omitted feature.

For example, according to an example embodiment of the present invention, the modified operating strategy for the selected feature can comprise at least one of the following procedures. For example, the gas sensor is no longer controlled for detecting measured values for the selected feature, i.e., the feature in question is omitted. Or no more measured values are detected for the selected feature. Or no more measured values for the selected feature are further processed or transmitted for further processing. Or a control of the gas sensor for detecting measured values for the selected feature is changed, e. g., to a different measurement time point.

In general, the measured values can, for example, be evaluated using a cluster analysis (possibly preceded by a principal component analysis). It may be that previously envisaged odors or odor types do not occur. In this case, as mentioned, the correlation analysis is performed, in which the base number of sensor features are examined at least in pairs (generally in tuples) and quantified, for example, by correlation coefficients. For example, if a first feature occurs in correlation with a second feature, both features contain equivalent information. The first feature can also only comprise a correlation as a result of a combination of the second feature with a third (or fourth, etc.) feature. The triple (or four-tuple, etc.) of features then contains only two (or three, etc.) independent pieces of information, and the third (or fourth, etc.) piece of information results from the other two (or three, etc.) pieces of information.

Both the cluster analysis and the correlation analysis can adequately appreciate the number of rare exceptions and consider them in the context of expectations. For example, a missing cluster may be present due to a few exceptional measurement points, or the correlation curve may show a few outliers from a systematic relationship.

For example, if it is expected that an unacceptable level of contamination is caused in 0.1% of all cases of using a vehicle interior, measured values corresponding to 0.1% are not outliers that justify omitting the feature. On the other hand, if contamination is expected in 50% of all cases, the 0.1% would be a candidate for an event that actually occurs too rarely. In the case of rare events, whether a feature can be omitted is thus also an economic decision, for example. The corresponding acceptance thresholds can, for example, be specified by the user (e. g., operator of a vehicle fleet).

According to an example embodiment of the present invention, the analysis of the measured values can be carried out in the central unit, e. g., in the so-called cloud or in another computing system or computing unit. The analysis can be performed, for example, for all of the plurality of the gas sensors or for a random sample. It can also be broken down according to systematic parameters, e.g., due to a limitation of the variability of the gas sensors, variability of the odor-forming substance or variability of disturbing substances. This can comprise climatic properties broken down according to region and season (or ambient temperatures and air humidities that could affect the raw sensor signals). This can comprise inhomogeneous user behavior; for example, for the cigarette smoke target variable, individual cigarette brands may smell different, and the individual brands may be more or less popular in certain target markets or age groups. This can comprise regional variations in outdoor air quality, e.g., inner cities, industrial areas or agricultural areas.

This breakdown can result in an evolutionary splitting of operating modes into a certain number of groups. For example, a first group can continue to work with the full base number of features. For a second group, one of the features can be omitted. For a third group, a different feature can be used, for example. In general, the modified operating strategy can thus, for example, only be used for a selected portion of the plurality of the similar gas sensors.

By repeatedly applying the proposed procedure, two or more of the base number of features can also be omitted for individual groups or in general.

As mentioned above, the modified operating strategy can comprise that measured values for a particular feature are not detected (omission of the feature). The modified operating strategy can also comprise that no more measured values for the particular or selected feature are further processed or transmitted for further processing. For example, in this case, a full measurement with the base number of features is carried out, but a reduced transmission of one less feature.

If a feature is omitted, switching off a raw value can mean that a measured value does not need to be detected, temporarily stored or transmitted in the gas sensor. However, in the case of thermally modulated metal oxide sensors, this does not necessarily mean that reaching a certain (intermediate) temperature can also be omitted. If the quality of the remaining features is influenced by a previous (intermediate) temperature, the temperature profile must be run unchanged over time.

On the other hand, if there are completely separate sensors/receptor layers of a physical sensor array and a single sensor or a single receptor layer exclusively provides the feature to be deleted, its heating element can be switched off.

As mentioned above, the control of the gas sensor for detecting measured values for the selected feature can also be changed. If, according to the considerations described above, a previous readout point (feature) of the gas sensor were a candidate for omitting this feature, a variation could also be carried out instead of the omission. For example, with an existing temperature profile, temperature gradations can be reached in stages or with slow gradients. For each point (feature), it is possible to read it earlier, later or at a different temperature in order nevertheless to increase the information content.

In one example embodiment of the present invention, the measurement number is at least one greater than the base number. In this case, if the result of the check is negative, i.e., if there is no correlation, the modified operating strategy detects measured values for the measurement number of features.

For example, if there is an over-the-air configuration option on the gas sensor hardware, the procedure for checking whether a feature can be omitted can also be carried out in reverse. For example, an additional feature is thus read out or generated.

When generating an additional feature, the above comments apply equally to thermally modulated gas sensors, i.e., a changed temperature profile for generating a new measured value or measuring point must not adversely affect the other readout points. The following options are therefore preferred. The temperature profile of the gas sensor remains unchanged. However, another readout point or measuring point on the receptor layer is placed temporally between two previously read readout points. If individual heating pulses have previously been far apart in time (e.g., a pulse profile with a duration of 10 seconds that is repeated periodically once per minute), the additional readout point with, if necessary, a new temperature can be placed directly at the end of the previous profile. The new profile with the new readout point or operating point and the previous profile with the base number of operating points (for the features) can be distributed into two test groups across the plurality of gas sensors. If no statistical difference can be recognized or proven across both groups in the base number of features or their measured values, it can be assumed that the new feature is neutral with respect to the previous features and the use of the new feature is possible, without limiting the accuracy of the previous machine learning model with respect to the previous features.

However, according to an example embodiment of the present invention, it may not be sufficient to simply prove the neutrality of the existing features (base number) if a new feature is introduced. For example, it should also be proven or shown that the new feature provides additional information, i.e., it must provide, in the existing correlation of pairs of sensor features or by clustering, additional points that correspond to outlier points. Which odor is present and whether it is a uniform odor class or a plurality of odors can then be determined, for example, by selecting the timestamps and identifying the gas sensors concerned (e.g., in vehicles), e.g., via a user survey. Accordingly, a new machine learning model can be trained with the new feature, wherein the data for which an annotation of the odor is available (e.g., from the aforementioned exemplary user survey) can be used as additional training data.

In summary, all features (base number) of the plurality, e. g., multiple thousands, of gas sensors can thus, for example, be examined together in one observation phase. If it turns out that at least one of the base number of features is not necessary, it can be “switched off” in the future, e.g., by an over-the-air update, i.e., the modified operating strategy is used. This is in particular the case if a correlation analysis can show that one of the base number of features (considered across a plurality of gas sensors) can also be predicted by one less than the base number of the features and is therefore not an independent measured value. However, a feature can also be omitted, for example, if it is proven to be above or below a measurement range limit of the sensor in the target application (so-called clipping).

The reason for a lower variety in comparison to the expected and developed features may, for example, be that not all target odors occur in reality (e.g., a cigarette odor because a smoking ban is observed), or (target) odors occur only in lower concentrations than are relevant for monitoring, or disturbances taken into account in the development phase and to be compensated have a lower variety in the application than was assumed in a development phase. For example, compensation for temperature and humidity fluctuations can be provided, but the conditions at the site of use turn out to be less fluctuating.

In order to avoid having to train a new machine learning model for the number of features now reduced by one, the omitted feature is, for example, emulated on the basis of the correlations and the previous model, which requires the base number of input variables, continues to be used.

According to an example embodiment of the present invention, by sampling the plurality of gas sensors (e. g., 1%) with the full base number of features without switch-off, an optional monitoring phase can check whether the omission still makes sense. In other words, once the modified operating strategy has been determined, it is thus checked whether the modified operating strategy is still appropriate. For example, typical user behavior could change again or fluctuations in temperature and humidity could occur again due to seasons or expanded sites of use.

Conversely, the method of the present invention can also be used to add an additional feature (as a candidate, so to speak) in order to identify newly measurable odors. Alternatively, instead of omitting or adding a feature, the existing feature could be varied. For example, a change can be made by varying a temperature level or a holding time of the temperature until a feature is read out or detected.

Repeating the method by switching off, adding and varying raw signal features can result in an evolutionary optimization of an operating profile of, for example, a thermally modulated gas sensor. The result may also be (depending on systematic variations in operating conditions) that operating modes are “split” depending on the application.

In one example embodiment of the present invention, a gas sensor is operated with a modified operating strategy, which was determined according to one of the above variants, and by using a machine learning model trained for the regular operating strategy. This is in particular true if the operation of the gas sensor for a feature is changed. On the other hand, in the case of an additional feature, it is advantageous to use a machine learning model that is trained for the modified operating strategy. Preferably, this operation comprises providing measured values detected by means of the gas sensor according to the modified operating strategy in a test environment. Based on the machine learning model and the measured values, a target substance in the target environment is then determined and information about the target substance is provided.

A computing unit according to the present invention, e. g., a control unit of a gas sensor or a computer, is configured, in particular programmatically, to perform a method according to the present invention. A gas sensor according to the invention comprises such a computing unit.

Furthermore, the implementation of a method according to the present invention in the form of a computer program or computer program product having program code for carrying out all the method steps of the method of the present invention is advantageous because it is particularly low-cost, in particular if an executing control unit is also used for further tasks and is therefore present anyway. Finally, a machine-readable storage medium is provided with a computer program as described above stored thereon. Suitable storage media or data carriers for providing the computer program are, in particular, magnetic, optical, and electric storage media, such as hard disks, flash memory, EEPROMs, DVDs, and others. It is also possible to download a program via computer networks (Internet, intranet, etc.). Such a download can be wired or wireless (e. g., via a WLAN network or a 3G, 4G, 5G or 6G connection, etc.).

Further advantages and embodiments of the present invention can be found in the description and the figures.

The present invention is shown schematically in the figures on the basis of exemplary embodiments and is described below with reference to the figures.

### BRIEF DESCRIPTION OF THE DRAWINGS

FIG. 1 schematically shows a gas sensor for explaining the present invention.

FIG. 2 schematically shows an arrangement for explaining the present invention.

FIGS. 3A, 3B schematically show sequences of the method according to the present invention in various preferred embodiments.

FIGS. 4, 5, 6 schematically show diagrams for explaining the present invention.

### DETAILED DESCRIPTION OF EXAMPLE EMBODIMENTS

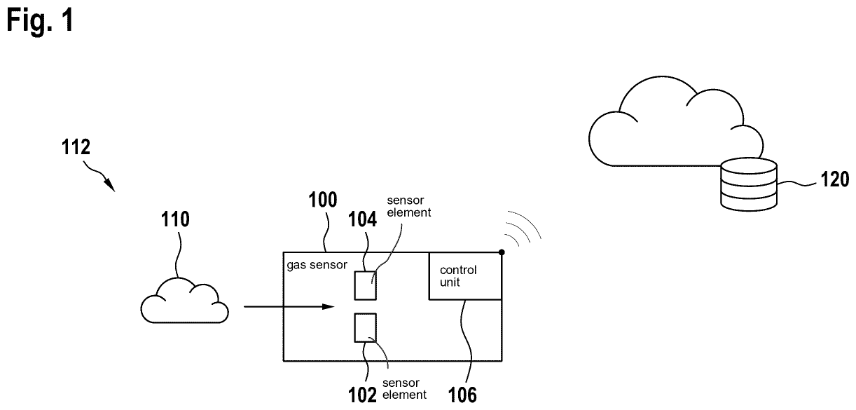

FIG. 1 schematically shows a gas sensor 100 for explaining the invention. By way of example, the gas sensor 100 comprises two sensor elements 102, 104, i.e., the sensor is a multi-gas sensor. Furthermore, the gas sensor 100 comprises a computing or control unit 106, on which, for example, operating software can run in order to detect and, if necessary, process measured values. By way of example, the control unit 106 is also configured for wireless communication in order, for example, to send data to a higher-level server 120 (e. g., in a so-called cloud) and/or to receive data from there.

Furthermore, a target substance 110 (i.e., for example, an odor source) in a target environment 112 is indicated by way of example, which target substance 110 can be recognized by means of the gas sensor 100 or about which information can be determined by using the gas sensor 100.

The gas sensor 100 shown in FIG. 1 can be used for an application for recognizing whether a target substance or odor source is present in the target environment, but also for collecting or generating training data. However, a comparable or different gas sensor can also be used for generating the training data.

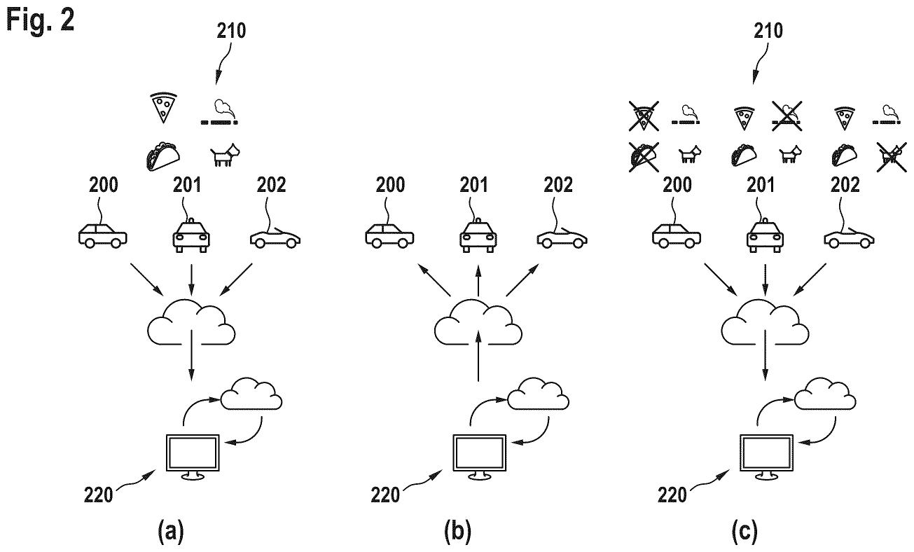

In FIG. 2, by way of example, illustrations (a), (b), (c) in each case show different vehicle types 200, 201, 202 as well as a higher-level server 220 (e.g., in a so-called cloud). By way of example, this is to represent a vehicle fleet, which in practice can comprise a plurality, e.g., thousands, of vehicles and comprises variants that in turn correspond to different typical user behavior. A gas sensor such as the gas sensor 110 according to FIG. 1 can be arranged or installed in each vehicle in order to detect odors in the corresponding vehicle.

The measured data detected by the gas sensors can, as shown in illustration (a), be transmitted to the higher-level server 220 via an indicated wireless communication link, e.g., mobile radio, as also explained in FIG. 1. For this purpose, by way of example, various odors 210 in the sense of target substances are also indicated as symbols. The gas sensors can be configured for detecting all of these different odors, namely, according to a regular operating strategy.

Based on an analysis, a modified operating strategy can now be determined, which is then transmitted to the gas sensors in the vehicles, as indicated in illustration (b). Then, for example, only certain odors are still detected, as indicated in illustration (c).

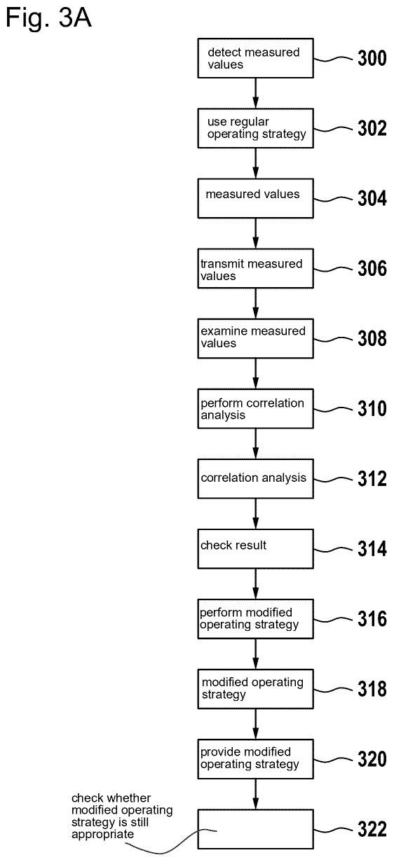

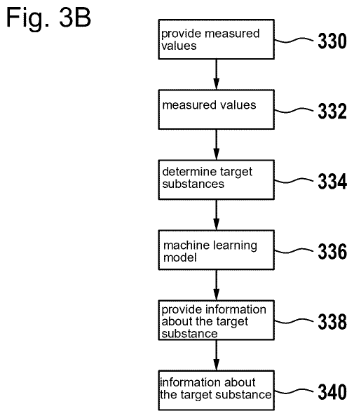

FIG. 3A schematically shows a sequence of a method in one embodiment, namely, for determining a modified operating strategy for a plurality of similar gas sensors. Each of the plurality of similar gas sensors in each case is configured to detect measured values for a base number of features according to a regular operating strategy. These gas sensors can be gas sensors as shown in FIG. 1, which are provided, for example, in vehicles as shown in FIG. 2 or in other devices.

For this purpose, step 300, measured values 304 are detected by each of the plurality of similar gas sensors using the regular operating strategy 302 in each case. These measured values are transmitted, step 306, to a higher-level server or other computing unit, where they are received and provided, as also shown, for example, in FIG. 2 illustration (a). Each gas sensor detects measured values for, for example, ten features or sensor features. In one embodiment, this number ten represents the base number and the measurement number.

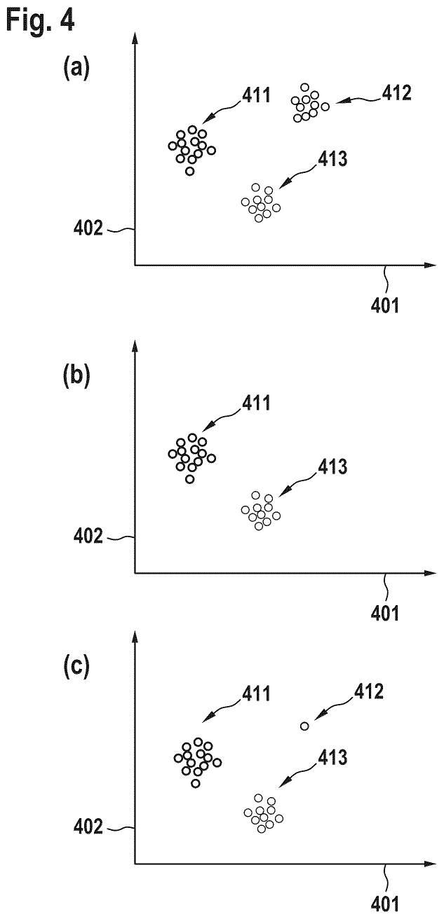

In an optional step 308, the measured values are then examined, for example, as to whether one of the ten features is not necessary. For this purpose, diagrams (a), (b), (c) in FIG. 4 show a cluster analysis. Such a cluster analysis can be carried out, for example, by means of a so-called principal component analysis. In the diagrams, in each case a first principal component 401 is plotted to the right and a second principal component 402 is plotted upward.

Diagram (a) shows that three clusters are formed, which can be assigned to an odor 411, 412, 413 in each case. Diagram (b) shows that the cluster for odor 412 is not present; this means that the odor 412 in question was not present in the underlying measured data. Diagram (c) shows that the cluster for odor 412 is only present with individual measured values; this may mean that the odor 412 in question was present significantly less frequently in the underlying measured data than expected. In case (b), but possibly also in case (c), it can then be decided that one of the ten features is not necessary or at least could be unnecessary or superfluous.

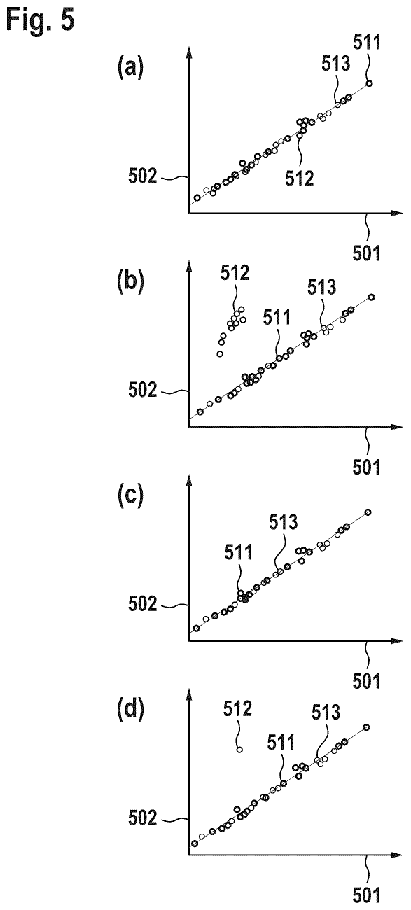

If one of the ten features is not necessary, a correlation analysis 312 is performed, step 310. However, as mentioned, the cluster analysis is optional. If the cluster analysis, i.e., step 308, is not performed, the correlation analysis, step 312, can be performed directly after step 306. The correlation analysis is performed between the measured values of, for example, two of the ten features in each case. Based on a result of the correlation analysis, it is then checked, step 314, whether a first of the ten features is correlated with a second of the ten features, at least within specified tolerances. In general, however, a linear relationship is not absolutely necessary for a correlation and a correlation may only be discovered by including more than two features. For illustration purposes, the simple case of correlation in pairs is shown in FIG. 5.

For this purpose, diagrams (a), (b), (c), (d) in FIG. 5 show such a correlation analysis or their result. In the diagrams, in each case a first feature or its raw signal 501 is plotted to the right and a second feature or its raw signal 502 is plotted upward. The higher the value of the raw signal, the higher the corresponding odor intensity, for example.

Diagram (a) shows that the measured values 511, 512, 513 for three different odors or odor types all lie on one line. For all measured values, the measured values of the features 501, 502 correlate. One of the two features can thus be omitted, for example.

Diagram (b) shows that the measured values 512 for one of the three odors or odor types do not lie on one line with the others. For the odors or odor types 511 and 513, the two features correlate and could be omitted. On the other hand, for the odor or odor type 512, this correlation does not exist. Thus, if odor or odor type 512 is to be recognized, neither of the two features should be omitted.

On the other hand, if odor or odor type 512, for example, does not appear in the measured values, as shown in diagram (c), one of the two features could be omitted, for example.

If odor or odor type 512 only appears sporadically in the measured values, as shown in diagram (d), a decision could be made, depending on the situation, whether one of the two features should be omitted.

In step 316, depending on a result of the check, a modified operating strategy 318 for the gas sensors is determined. In particular, this comprises that an operation for a selected one of the first and the second of the ten features is or is being changed.

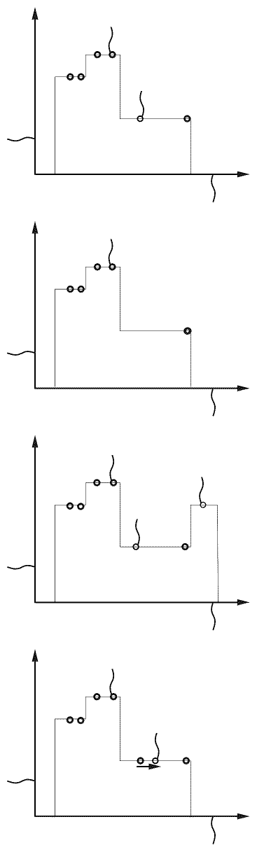

For this purpose, diagrams (a), (b), (c), (d) in FIG. 6 show operating strategies. In the diagrams, in each case a temperature 602 is plotted over time 601. The curves shown in the diagrams correspond to a temperature profile, i.e., the temperature level is, for example, changed multiple times and kept constant for a certain time period in each case. The points indicate time points for detecting a measured value and thus a feature; by way of example, features 611, 612, 613 are denoted in more detail in the diagrams and are discussed below.

By way of example, diagram (a) shows six features, including features 611, 612. The operating mode here can be a regular operating mode, for example. By way of example, diagram (b) shows only five features, wherein the feature 611 is still present, but the feature 612 is not, i.e., it has been omitted. The operating strategy here can thus be a modified operating strategy.

By way of example, diagram (c) shows seven features, wherein the features 611, 612 are still present, as is an additional feature 613. At the end of the temperature profile of the regular operating strategy, a further temperature level is added at which the measured value 613 (as a feature) is detected. The operating strategy here can thus also be a modified operating strategy.

By way of example, diagram (d) shows six features, wherein the features 611, 612 are still present, but the detection time point of the feature 612 has been postponed to later. The operating strategy here can thus also be a modified operating strategy.

The modified operating strategy is then provided, step 320, for use with the gas sensors, as also shown in FIG. 2 illustration (b), for example. In a step 322, it can then be checked whether the modified operating strategy is still appropriate.

FIG. 3B schematically shows a sequence of a method in one embodiment, namely, for operating a gas sensor with a modified operating strategy, which has been determined, for example, as shown in FIG. 3A, by using a machine learning model that has been trained for the regular operating strategy.

In this case, step 330, measured values 332 are provided, which have been detected by means of the gas sensor in a target environment according to the modified operating strategy. In step 334, based on a machine learning model 336 and the measured values, a target substance in the target environment is determined; then, step 338, information 340 about the target substance is provided.

For the machine learning model, which is, for example, trained on ten features and therefore requires measured values for ten features, only measured values for nine features are, for example, detected or provided; the measured value for the tenth feature can, as mentioned, be emulated, for example.

1-16. (canceled)

17. A method for determining a modified operating strategy for a plurality of similar gas sensors, wherein each of the plurality of similar gas sensors is configured to detect measured values for a base number of features according to a regular operating strategy, the method comprising the following steps:
providing measured values detected by the plurality of similar gas sensors with a measurement number of features;
performing a correlation analysis between the measured values of at least two of the measurement number of the features in each case;
checking, based on a result of the correlation analysis, whether a first of the measurement number of the features is correlated with at least a second of the measurement number of the features, at least within specified tolerances;
depending on a result of the check, determining a modified operating strategy for the plurality of similar gas sensors; and
providing the modified operating strategy for use with the plurality of similar gas sensors.

18. The method according to claim 17, wherein the similar gas sensors are based on a thermally modulated sensor, wherein each gas sensor is configured to run through a specified temperature profile in the regular operating strategy and, for various features:
to detect measured values at different temperatures, and/or to detect measured values at different time periods since a change in temperature.

19. The method according to claim 17, further comprising:
examining the measured values as to: (i) whether one of the base number of the features of the plurality of similar gas sensors is redundant and thus not necessary, or (ii) whether there are indications that at least one additional feature could provide additional information;
wherein the correlation analysis is performed only when the examination is positive.

20. The method according to claim 17, wherein the measurement number corresponds to the base number, and wherein, when the result of the check is positive, in the modified operating strategy for the plurality of similar gas sensors, an operation for a selected one of the first and the at least one second of the measurement number of the features is changed.

21. The method according to claim 20, wherein the modified operating strategy for the selected feature includes at least one of the following procedures:
a gas sensor is no longer controlled for detecting measured values for the selected feature,
no more measured values for the selected feature are detected,
no more measured values for the selected feature are further processed or transmitted for further processing,
a control of the gas sensor for detecting measured values for the selected feature is changed.

22. The method according to claim 17, wherein the measurement number is at least one greater than the base number, and wherein, when the result of the check is negative, measured values for the measurement number of features are detected in the modified operating strategy.

23. The method according to claim 22, wherein the modified operating strategy includes use of a machine learning model trained for the modified operating strategy.

24. The method according to claim 17, wherein the modified operating strategy includes use of a machine learning model trained for the regular operating strategy.

25. The method according to claim 17, wherein the modified operating strategy is used only for a selected portion of the plurality of similar gas sensors.

26. The method according to claim 17, wherein, after the modified operating strategy has been determined, it is checked whether the modified operating strategy is still required.

27. A method for operating a gas sensor with a modified operating strategy by using a machine learning model trained for a regular operating strategy or trained for the modified operating strategy, the modified operating strategy being determined for a plurality of similar gas sensors, wherein each of the plurality of similar gas sensors is configured to detect measured values for a base number of features according to a regular operating strategy, the modified operating strategy being determined by:
providing measured values detected by the plurality of similar gas sensors with a measurement number of features;
performing a correlation analysis between the measured values of at least two of the measurement number of the features in each case;
checking, based on a result of the correlation analysis, whether a first of the measurement number of the features is correlated with at least a second of the measurement number of the features, at least within specified tolerances;
depending on a result of the check, determining a modified operating strategy for the plurality of similar gas sensors; and
providing the modified operating strategy for use with the plurality of similar gas sensors.

28. The method according to claim 27, further comprising:
providing measured values detected using the gas sensor in a target environment according to the modified operating strategy;
determining, based on the machine learning model and the measured values from the gas sensor, a target substance in the target environment; and
providing information about the target substance.

29. A processing unit configured to determine a modified operating strategy for a plurality of similar gas sensors, wherein each of the plurality of similar gas sensors is configured to detect measured values for a base number of features according to a regular operating strategy, the processing unit configured to perform the following steps:
providing measured values detected by the plurality of similar gas sensors with a measurement number of features;
performing a correlation analysis between the measured values of at least two of the measurement number of the features in each case;
checking, based on a result of the correlation analysis, whether a first of the measurement number of the features is correlated with at least a second of the measurement number of the features, at least within specified tolerances;
depending on a result of the check, determining a modified operating strategy for the plurality of similar gas sensors; and
providing the modified operating strategy for use with the plurality of similar gas sensors.

30. A gas sensor with a computing unit configured to operate the gas sensor with a modified operating strategy by using a machine learning model trained for a regular operating strategy or trained for the modified operating strategy, the modified operating strategy being determined for a plurality of similar gas sensors, wherein each of the plurality of similar gas sensors is configured to detect measured values for a base number of features according to a regular operating strategy, the modified operating strategy being determined by:
providing measured values detected by the plurality of similar gas sensors with a measurement number of features;
performing a correlation analysis between the measured values of at least two of the measurement number of the features in each case;
checking, based on a result of the correlation analysis, whether a first of the measurement number of the features is correlated with at least a second of the measurement number of the features, at least within specified tolerances;
depending on a result of the check, determining a modified operating strategy for the plurality of similar gas sensors; and
providing the modified operating strategy for use with the plurality of similar gas sensors.

31. A non-transitory machine-readable storage medium on which is stored a computer program for determining a modified operating strategy for a plurality of similar gas sensors, wherein each of the plurality of similar gas sensors is configured to detect measured values for a base number of features according to a regular operating strategy, the computer program, when executed by a computer, causing the computer to perform the following steps:
providing measured values detected by the plurality of similar gas sensors with a measurement number of features;
performing a correlation analysis between the measured values of at least two of the measurement number of the features in each case;
checking, based on a result of the correlation analysis, whether a first of the measurement number of the features is correlated with at least a second of the measurement number of the features, at least within specified tolerances;
depending on a result of the check, determining a modified operating strategy for the plurality of similar gas sensors; and
providing the modified operating strategy for use with the plurality of similar gas sensors.
